(19) United States
(12) Patent Application Publication
(10) Pub. No.: US 2023/0317440 A1
(43) Pub. Date: Oct. 5, 2023

(54) METHOD FOR DISCRIMINATING ORE PROSPECTING TYPES BASED ON COMPOSITIONAL CHANGE OF EPIDOTE

(71) Applicants: TIBET JULONG COPPER CO., LTD., Lhasa (CN); CHINA UNIVERSITY OF GEOSCIENCES, BEIJING, Beijing (CN)

(72) Inventors: YOUYE ZHENG, BEIJING (CN); SONG WU, BEIJING (CN); JIANHUI WU, LHASA (CN); YIBIN LIN, LHASA (CN); DECAI LIN, LHASA (CN); XIANWU SHA, LHASA (CN); XIAOFANG DOU, BEIJING (CN); CAIXIAN CHEN, LHASA (CN); JIANCHANG LI, LHASA (CN); YAMING ZHANG, LHASA (CN); GUANGWU JIANG, LHASA (CN)

(21) Appl. No.: 18/122,128

(22) Filed: Mar. 16, 2023

(30) Foreign Application Priority Data
Mar. 18, 2022 (CN) 202210273006.0

(51) Int. Cl. H01J 49/00; G01N 33/24; H01J 49/10; G01N 1/28

(52) U.S. Cl. CPC H01J 49/0036; G01N 1/286; G01N 33/24; H01J 49/105; G01N 2001/2866

(57) ABSTRACT

Disclosed is a method for discriminating ore prospecting types based on compositional change of epidote, in particular including the following steps: metallogenic zone delineation; sample collection and analysis: collecting bedrock samples containing epidote from the metallogenic zone according to certain sampling units; trace element analysis and testing; data processing and interpretation: processing obtained initial recorded data using LADRlib software; and ore prospecting type discrimination. The method has the advantages that the description of epidote altered minerals in a magma-hydrothermal metallogenic system is improved from macroscopic characterization to microscopic quantitative interpretation of trace element change in the epidote altered minerals by using an LA-ICP-MS in-situ analysis technology, and the trace element change is linked with the response of the ore prospecting type at an ore concentration area scale, thus overcoming the difficulties of low efficiency, long period and high cost in the traditional method for discriminating the ore prospecting type.

### FIELD OF TECHNOLOGY

The present invention relates to the technical field of ore prospecting in geological exploration, and in particular to a method for discriminating ore prospecting types based on compositional change of epidote.

### BACKGROUND

With the comprehensive coverage of geological exploration in recent decades, the degree of mineral exploration has gradually increased, the discovery of new deposits has become increasingly difficult, and there is an urgent need to carry out new ore prospecting technical methods to guide the ore prospecting breakthrough, of which the primary task is to quickly discriminate the ore prospecting types.

The traditional discrimination of ore prospecting types has the following shortcomings: comprehensive research, such as large-scale mapping and systematic sampling analysis, is required before exploration and evaluation to clarify the orebody occurrence, the relationship with wall rocks, metallogenesis, ore-forming materials and ore-forming fluid sources, etc., so as to determine the genesis of deposits or ore prospecting types, resulting in long period and high cost, which cannot meet the urgent need for rapid exploration and evaluation at the ore concentration area scale.

### SUMMARY

To solve the technical problem above, the present invention provides a method for discriminating ore prospecting types based on compositional change of epidote. The method provided by the present invention has the advantages that the description of epidote altered minerals in a magma-hydrothermal metallogenic system is improved from macroscopic characterization to microscopic quantitative interpretation of trace element change in the epidote altered minerals by using an advanced LA-ICP-MS (laser ablation inductively coupled plasma mass spectrometry) in-situ analysis technology, and the trace element change is linked with the response of the ore prospecting type at an ore concentration area scale, thus overcoming the difficulties of low efficiency, long period and high cost in the traditional method for discriminating the ore prospecting types.

The present invention is achieved through the following technical solutions: a method for discriminating ore prospecting types based on compositional change of epidote specifically includes the following steps:
metallogenic zone delineation: conducting data collection according to a selected research area, and comprehensively analyzing metallogenic potential to delineate a metallogenic favorable zone;
sample collection and analysis: collecting bedrock samples containing epidote from the metallogenic zone according to certain sampling units in step (1), where the density of sample points collected in the research area is 1-3 samples/Km²;
trace element analysis and testing: grinding the samples collected in step (2) into a probe sheet and a laser in-situ target, firstly observing corresponding epidote alteration characteristics of the probe sheet and the laser in-situ target under a microscope, and recording epidote alteration types in detail; then performing electron probe compositional analysis to determine the chemical composition and type of the epidote, and marking the chemical composition and type of the epidote; and carrying out in-situ microdomain element analysis of laser ablation inductively coupled plasma mass spectrometry (LA-ICPMS) on the mineral which is confirmed as the epidote by the electron probe composition, so as to obtain recorded data of each test point;
data processing and interpretation: processing the initial recorded data obtained in step (3) by using LADRlib software;
(5) ore prospecting type discrimination: processing data obtained in step (4) by using Excel, defining the contents of La, Y, Gd, Yb, Sr and As elements in the obtained epidote as V_{(La)}, V_{(Y)}, V_{(Gd)}, V_{(Yb)}, V_{(Sr)} and V_{(As)}, respectively, and substituting the content of La element in the obtained epidote into the following formula (1):
$$\text{C1=0} \text{.28493059*lg} \left({\text{V}}_{\left(\text{La}\right)}\right) \text{+0} \text{.5762992}$$
calculating a discriminant factor C1, when C1 is greater than lg(V_{(Y)}), discriminating that a deposit to which the epidote belongs is a porphyry type deposit; and when C1 is less than 1 g(V_{(Y)}), discriminating that a deposit to which the epidote belongs is an epithermal type deposit; and
substituting the contents of Gd and Yb elements in the obtained epidote into the following formula (2):
$$\text{C2=lg} \left({\text{V}}_{\left(\text{Gd+Yb}\right)}\right) \text{+1} \text{.5}$$
calculating a discriminant factor C2, when C2 is less than lg(V_{(Sr/As)}), discriminating that a deposit to which the epidote belongs is a porphyry type deposit; and when C2 is greater than lg(V_{(Sr/As)}), discriminating that a deposit to which the epidote belongs is an epithermal type deposit.

Through the technical solution above, geochemical characteristics of the epidote are introduced into the discrimination of ore prospecting types, which effectively makes up for the defect that there is no quantitative discrimination method at present, and the calculation method is more novel. Further, in step (5), the formula (1) is to perform logarithm transformation on the contents of La and Y elements in the epidote to obtain lg(V_{(La)}) and lg(V_{(y)}), to plot with lg(V_{(La)}) as the abscissa and lg(V_{(Y)}) as the ordinate, respectively, and to obtain a demarcation line between the porphyry type deposit and epithermal type deposit based on the plotting range.

Further in step (5), the formula (2) is to perform logarithm transformation on the contents of Gd, Yb, Sr and As elements in the epidote to obtain lg(V_{(Gd+Yb)}) and lg(V_{(Sr/As)}), to plot with lg(V_{(Gd+Yb)}) as the abscissa and lg(V_{(Sr/As)}) as the ordinate, respectively, and to obtain a demarcation line between the porphyry type deposit and epithermal type deposit based on the plotting range.

Further, step (2) includes the following steps: positioning each sample point by using a GPS positioning system, collecting coordinate data X and Y, taking field photos, and making detailed field records to describe lithology, alteration and mineralization characteristics of each sample.

Further, in step (4), the specific process is as follows:
(1) data import: importing the recorded data in a csv format obtained from an in-situ microdomain test point of each epidote sample into the LADRlib software in batches;
(2) data interpretation: obtaining a microdomain element integral curve of the sample at each observation point, and adjusting start time and end time of the integral curve of each observation point one by one according to the principle of ensuring the flattest and widest signal range of the selected element integral curve;
(3) data filtration: rejecting invalid data therein according to anomaly peaks of the element integral curve; and
(4) data export: summarizing interpreted and filtered data of each single-point microdomain, and then exporting the data in batch as a file in a csv format.

Through the technical solution above, the influence of other auxiliary minerals on the calculation of the discriminant factors of trace elements in the epidote mineral can be eliminated, and the method may be suitable for the processing of trace element data of the epidote in different types of deposits.

Preferably, the research area is Zhunuo ore concentration area.

In addition, application of a method for discriminating ore prospecting types based on compositional change of epidote for the discrimination of an epithermal type Ag-Au deposit is provided. Quantitative indexes of trace elements in the epidote are as follows: calculating discriminant factors C1=0.28493059*lg(V_{(La)})+0.5762992 and C2=lg(V_{(Gd+Yb)})+ 1.5, respectively, according to the formula (1) and the formula (2); and when C1 is less than lg(V_{(Y)}) and C2 is greater than lg(V_{(Sr+As)}), discriminating that a deposit to which the epidote belongs is an epithermal type deposit.

Application of a method for discriminating ore prospecting types based on compositional change of epidote for the discrimination of a porphyry type Cu deposit is provided. Quantitative indexes of trace elements in the epidote are as follows: calculating discriminant factors C1=0.28493059*lg(V_{(La)})+0.5762992 and C2=lg(V_{(Gd+Yb)})+ 1.5, respectively, according to the formula (1) and the formula (2); and when C1 is greater than lg(V_{(Y)}) and C2 is less than lg(V_{(Sr+As)}), discriminating that a deposit to which the epidote belongs is a porphyry type deposit.

Through the technical solution above, the present invention belongs to a microscopic quantitative discrimination method, which is more scientific, reasonable and accurate in comparison with the traditional macroscopic qualitative discrimination method.

Compared with the prior art, the present invention has the advantages that:

(1) The description of epidote altered minerals in a magma-hydrothermal mineralization system is improved from macroscopic characterization to microscopic quantitative interpretation of trace element changes by using an advanced LA-ICP-MS in-situ analysis technology, and the trace element change is linked with the response of the ore prospecting types in an ore concentration area scale, which effectively makes up for the defect that there is no quantitative discrimination method at present, and overcomes the difficulties of low efficiency, long period and high cost in the traditional method for discriminating the ore prospecting types.

(2) It is proposed to use epidote as a characteristic mineral for discrimination, which has good connectivity and wide physical and chemical conditions, is sensitive to the changes of physical and chemical conditions, can be formed at high temperature, medium temperature and low temperature, is uniform in spatial distribution, develops in different alteration zones, and thus is more conducive to the distinction of different mineralization types.

(3) The characteristic elements, such as La, Y, Sr, As, Gd and Yb, in the epidote are sensitive to the changes of temperature, pH and redox conditions.

(4) An optimal discriminant factor is provided, which may be used for accurately distinguishing different types of deposits, and can provide a theoretical basis for the optimization and selection of further ore prospecting and exploration methods in the ore deposit scale and reduce the exploration risk.

### BRIEF DESCRIPTION OF THE DRAWINGS

FIG. 1 is a diagram illustrating ore prospecting type discrimination based on trace element change of epidote in accordance with the present invention;

FIG. 2 illustrates metallogenic favorable zone delineation and sample cell division based on hyperspectral remote sensing in the Zhunuo ore concentration area in accordance with the present invention;

FIG. 3 illustrates laser in-situ target and analysis testing of epidote in Zhunuo ore concentration area.

### DESCRIPTION OF THE EMBODIMENTS

The present invention is further described below with reference to the accompanying drawings.

A method for discriminating ore prospecting types based on compositional change of epidote specifically includes the following steps:
(1) Metallogenic zone delineation: data is collected according to a selected research area, and metallogenic potential is comprehensively analyzed, so as to delineate a metallogenic favorable zone.
(2) Sample collection and analysis: bedrock samples containing epidote are collected from the metallogenic zone according to certain sampling units in step (1), where the density of sample points collected in the research area is 1-3 samples/Km².
(3) Trace element analysis and testing: the samples collected in step (2) are ground into a probe sheet and a laser in-situ target, corresponding epidote alteration characteristics of the probe sheet and the laser in-situ target are firstly observed under a microscope, and epidote alteration types are recorded in detail. Then, electron probe compositional analysis is performed to determine the chemical composition and type of the epidote, and the chemical composition and type of the epidote are marked; and the mineral which is confirmed as the epidote by the electron probe composition is subjected to in-situ microdomain element analysis of laser ablation inductively coupled plasma mass spectrometry (LA-ICPMS), so as to obtain recorded data of each test point.
(4) Data processing and interpretation: the initial recorded data obtained in step (3) are processed by using LADRlib software, with specific process as follows:
   (1) data import: importing the recorded data in a csv format obtained from an in-situ microdomain test point of each epidote sample into the LADRlib software in batches;
   (2) data interpretation: obtaining a microdomain element integral curve of the sample at each observation point, and adjusting start time and end time of the integral curve of each observation point one by one according to the principle of ensuring the flattest and widest signal range of the selected element integral curve;
   (3) data filtration: rejecting invalid data therein according to anomaly peaks of the element integral curve; and
   (4) data export: summarizing interpreted and filtered data of each single-point microdomain, and then exporting the data in batch as a file in a csv format.
(5) Ore prospecting type discrimination: data obtained in step (4) are processed by using Excel, the contents of La, Y, Gd, Yb, Sr and As elements in the obtained epidote are defined as V_{(La)}, V_{(Y)}, V_{(Gd)}, V_{(Yb)}, V_{(Sr)} and V_{(As)}, respectively, and the content of La element in the obtained epidote is substituted into the following formula (1):
$$\text{C1=0} \text{.28493059*lg} \left({\text{V}}_{\left(\text{La}\right)}\right) \text{+0} \text{.5762992}$$
a discriminant factor C1 is calculated, when C1 is greater than lg(V_{(Y)}), it is discriminated that a deposit to which the epidote belongs is a porphyry type deposit; and when C1 is less than lg(V_{(Y)}), it is discriminated that a deposit to which the epidote belongs is an epithermal type deposit;
the contents of Gd and Yb elements in the obtained epidote are substituted into the following formula (2):
$$\text{C2=lg} \left({\text{V}}_{\left(\text{Gd+Yb}\right)}\right) \text{+1} \text{.5}$$
a discriminant factor C2 is calculated, when C2 is less than lg(V_{(Sr/As)}), it is discriminated that a deposit to which the epidote belongs is a porphyry type deposit; and when C2 is greater than lg(V_{(Sr/As)}), it is discriminated that a deposit to which the epidote belongs is an epithermal type deposit.

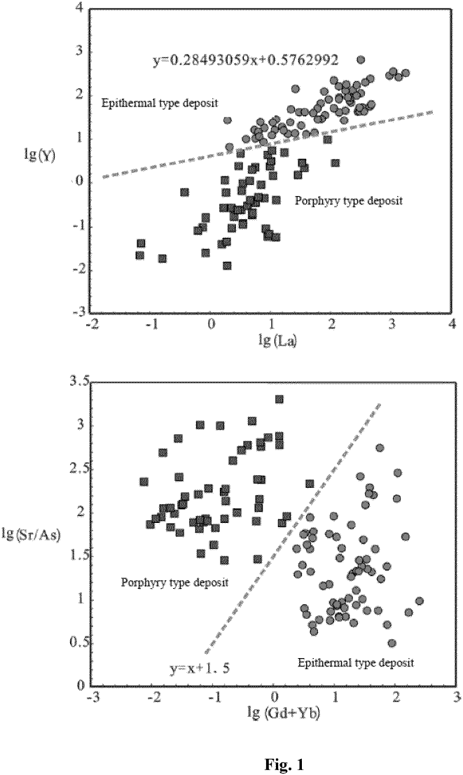

The specific steps are as follows:
step one, calculation of the discriminant factor C1
performing logarithm transformation on the contents of La and Y elements in the obtained epidote to obtain lg(V_{(La)}) and lg(V_{(Y)}), plotting with lg(V_{(La)}) as the abscissa and lg(V_{(Y)}) as the ordinate, respectively, obtaining a demarcation line between the porphyry type deposit and the epithermal type deposit based on the plotting range, and calculating the discriminant factor C1 by using the following formula:
$$\text{C1=0} \text{.28493059*lg} \left({\text{V}}_{\left(\text{La}\right)}\right) \text{+0} \text{.5762992}$$
step two, calculation of the discriminant factor C2
performing logarithm transformation on the contents of Gd, Yb, Sr and As elements in the obtained epidote to obtain lg(V_{(Gd+Yb)}) and lg(V_{(Sr/As)}); plotting with the lg(V_{(Gd+Yb)}) as the abscissa and the lg(V_{(Sr/As)}) as the ordinate, obtaining a demarcation line between the porphyry type deposit and the epithermal type deposit based on the plotting range, and calculating the discriminant factor C2 using the following formula:
$$\text{C2=lg} \left({\text{V}}_{\left(\text{Gd+Yb}\right)}\right) \text{+1} \text{.5}$$
step three, discrimination of the ore prospecting type
   substituting the content of La element in the obtained epidote into the formula above to calculate the discriminant factor C1, when C1 is greater than lg(V_{(Y)}), discriminating that a deposit to which the epidote belongs is a porphyry type deposit; and when C1 is less than lg(V_{(Y)}), discriminating that a deposit to which the epidote belongs is an epithermal type deposit (FIG. 1); and
   substituting the contents of Gd and Yb elements in the obtained epidote into the formula above to calculating the discriminant factor C2, when C2 is less than lg(V_{(Sr/As)}), discriminating that a deposit to which the epidote belongs is a porphyry type deposit; and when C2 is greater than lg(V_{(Sr/As)}), discriminating that a deposit to which the epidote belongs is an epithermal type deposit (FIG. 1).

On the basis of the above solution, step (2) includes the following steps: positioning each sample point by using a GPS positioning system, collecting coordinate data X and Y, taking field photos, and making detailed field records to describe lithology, alteration and mineralization characteristics of each sample.

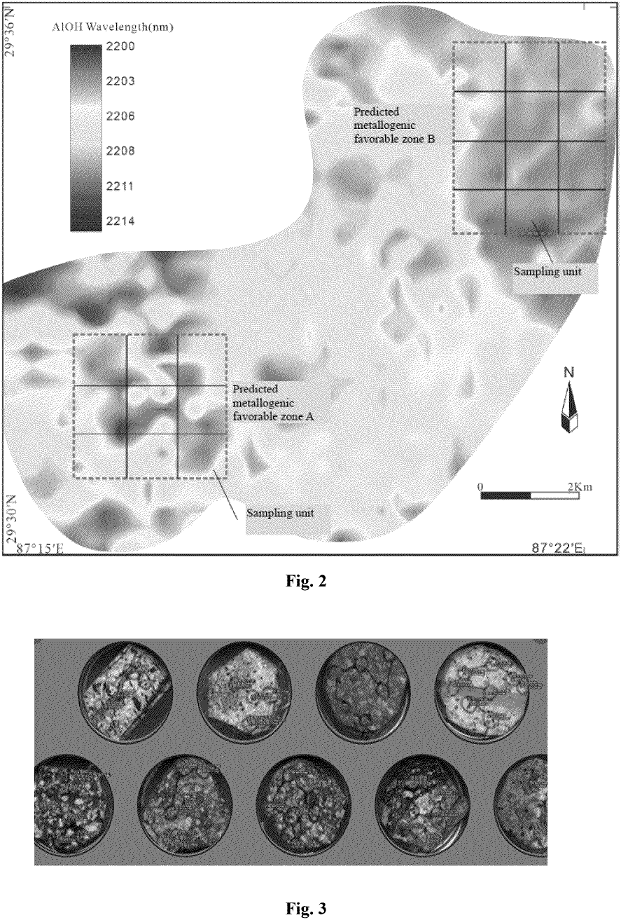

On the basis of the above solution, preferably, the research area is the Zhunuo ore concentration area, specifically as follows:
a. The existing geological, geophysical, geochemical and remote sensing data in the Zhunuo ore concentration area are collected systematically, the metallogenic potential is analyzed comprehensively, metallogenic favorable zones A and B are delineated, and sampling cells are marked off, where the spacing between the cells is 1 km×1 km, and the area is 1 km² (FIG. 2).
b. Field sample collection:
   The metallogenic favorable zones A and B are selected for the collection of epidote samples on the earth surface. During sampling, the sample number, sampling coordinates (X and Y), lithology category, alteration category, mineralization category and sampling site are recorded in detail, specifically as shown in the following table:

| Samplin g number | X | Y | Lithology | Hand specimen alteration | Minerali zation | Site |
|---|---|---|---|---|---|---|
| C04432 | 525897 | 326636 6 | Granodiorite porphyry | Quartz-epidote vein | Pyritizati on | A |
| 16334 | 534824 | 327424 0 | Monzonitic granite | Clumpy epidotization | Pyritizati on | B |
| ... | ... | ... | ... | ... | ... | ... |

c. Sample test:
   The collected samples are ground into a probe sheet and a laser in-situ target, corresponding epidote alternation characteristics of the probe sheet and the laser in-situ target are observed under a microscope, and epidote alternation types (hydrothermal vein type or disseminated type) of the probe sheet and the laser in-situ target are recorded in detail. Electron probe compositional analysis is performed to determine the chemical composition and type of the epidote, and the chemical composition and type are marked by using a marking pen. The mineral with the composition confirmed as the epidote by the electron probe testing is subjected to in-situ microdomain element analysis of laser ablation inductively coupled plasma mass spectrometry (LA-ICPMS) (FIG. 3).
d. Data processing: the LADRlib software is used for data processing, including four steps: (1) data import: importing the recorded data in a csv format obtained from an in-situ microdomain test point of each epidote sample into the LADRlib software in batches; (2) data interpretation: obtaining a microdomain element integral curve of the sample at each observation point, and adjusting start time and end time of the integral curve of each observation point one by one according to the principle of ensuring the flattest and widest signal range of the selected element integral curve; (3) data filtration: rejecting invalid data therein according to anomaly peaks of the element integral curve, e.g., the data such as hitting inclusions (Ti, Ag, As elements anomaly peaks) or hitting through epidote minerals (K, Mg elements anomaly peaks); and (4) data export: summarizing interpreted and filtered data of each single-point microdomain, and then exporting the data in batch as a file in a csv format.
e. Ore prospecting type discrimination:
   Final data (Table 1) processed by using Excel is used to discriminate that an ore prospecting zone B is an ore prospecting type of a porphyry type Cu deposit and an ore prospecting zone A is an ore prospecting type of an epithermal type Ag-Au deposit (FIG. 1) according to calculation results of the discriminant factors C1 and C2.

TABLE 1

| Sample number | R e g i o n | Y | La | Sr/A s | Gd+Y b | lg(Sr/A s) | lg (Gd+ Yb) | lg ( Y ) | lg (La ) | Discri minant factor C1 | Discri mina nt factor C2 |
|---|---|---|---|---|---|---|---|---|---|---|---|
| 49-C1-2-epi | A | 42.0 172 5 | 30. 34 14 | 6.49 4499 | 11.99 86851 | 0.8125 45624 | 1.0791 33656 | 1.62 3428 | 1.48 2035 | 0.9985 7616 | 2.579 1337 |
| 49-C1-3-epi | A | 16.2 514 | 11. 89 22 | 38.7 295 | 4.015 30393 | 1.5880 41836 | 0.6037 18424 | 1.21 0891 | 1.07 5261 | 0.8826 7366 | 2.103 7184 |
| 49-C1-4-epi | A | 16.1 909 9 | 5.5 04 99 | 8.03 0206 | 3.268 96537 | 0.9047 26704 | 0.5144 1032 | 1.20 9273 | 0.74 0756 | 0.7873 6312 | 2.014 4103 |
| 49-C2-3-epi | A | 28.3 235 3 | 12. 49 31 | 9.38 4982 | 9.676 7678 | 0.9724 33447 | 0.9857 3032 | 1.45 2147 | 1.09 6671 | 0.8887 7408 | 2.485 7303 |
| 49-C2-4-epi | A | 90.8 387 8 | 14 0.5 7 | 161. 7404 | 44.18 24568 | 2.2088 18463 | 1.6452 49861 | 1.95 8271 | 2.14 7893 | 1.1882 9954 | 3.145 2499 |
| 49-C3-1-epi | A | 66.6 641 7 | 68. 82 05 | 8.75 7943 | 23.03 84727 | 0.9424 0213 | 1.3624 53685 | 1.82 3892 | 1.83 7718 | 1.0999 2107 | 2.862 4537 |
| 49-C3-2-epi | A | 24.4 536 7 | 21. 43 49 | 7.38 6988 | 8.824 8657 | 0.8684 6739 | 0.9457 08105 | 1.38 8344 | 1.33 1121 | 0.9555 7608 | 2.445 7081 |
| 49-C3-3-epi | A | 48.5 033 1 | 22. 12 5 | 9.47 6942 | 13.38 5277 | 0.9766 6821 | 1.1266 27363 | 1.68 5771 | 1.34 4882 | 0.9594 9711 | 2.626 6274 |
| 49-C3-4-epi | A | 19.0 545 3 | 15. 69 48 | 5.96 0881 | 5.703 31272 | 0.7753 10444 | 0.7561 27185 | 1.27 9998 | 1.19 5756 | 0.9170 0657 | 2.256 1272 |
| 49-C4-1-epi | A | 20.4 064 | 32. 55 18 | 15.0 8144 | 8.331 8441 | 1.1784 42933 | 0.9207 41135 | 1.30 9766 | 1.51 2574 | 1.0072 7772 | 2.420 7411 |
| 49-C4-2-epi | A | 18.0 833 5 | 7.9 82 37 | 5.17 9454 | 4.402 43998 | 0.7142 84002 | 0.6436 93444 | 1.25 7279 | 0.90 2132 | 0.8333 4402 | 2.143 6934 |
| 49-C6-1-epi | A | 35.8 877 8 | 81. 30 35 | 39.5 1874 | 13.31 68845 | 1.5968 03124 | 1.1244 02631 | 1.55 4947 | 1.91 0109 | 1.1205 4756 | 2.624 4026 |
| 49-C6-2-epi | A | 164. 921 7 | 137.6 82 | 7.58 0657 | 53.20 07609 | 0.8797 06849 | 1.7259 17844 | 2.21 7278 | 2.13 8877 | 1.1857 3043 | 3.225 9178 |
| 49-C7-2-epi | A | 12.9 179 | 39. 70 74 | 61.3 4746 | 4.564 79013 | 1.7877 96595 | 0.6594 20815 | 1.111 192 | 1.59 8872 | 1.0318 6646 | 2.159 4208 |
| 49-C7-3-epi | A | 50.9 197 8 | 60. 96 05 | 10.5 2708 | 17.42 1238 | 1.0223 07754 | 1.2410 79013 | 1.70 6887 | 1.78 5048 | 1.0849 1389 | 2.741 079 |
| 53-C1-3-epi | A | 331. 647 6 | 17 67. 32 | 7.20 6871 | 168.0 3277 | 0.8577 46733 | 2.2253 93986 | 2.52 0677 | 3.24 7315 | 1.5015 5848 | 3.725 394 |
| 53-C2-1-epi | A | 26.8 252 9 | 16 8.6 06 | 8.57 8457 | 11.110 9012 | 0.9334 09184 | 1.0457 49286 | 1.42 8544 | 2.22 6872 | 1.2108 0293 | 2.545 7493 |
| 53-C2-4-epi | A | 61.8 892 4 | 47 4.1 37 | 7.57 0974 | 34.76 74826 | 0.8791 51759 | 1.5411 73246 | 1.79 1615 | 2.67 5903 | 1.3387 4575 | 3.041 1732 |
| 53-C2-5-epi | A | 43.1 073 7 | 28 4.7 38 | 8.15 5897 | 14.75 5438 | 0.9114 71719 | 1.1689 52106 | 1.63 4551 | 2.45 4446 | 1.2756 4576 | 2.668 9521 |
| 53-C5-3-epi | A | 69.4630 4 | 21 9.2 33 | 20.16176 | 20.0925576 | 1.3045 28507 | 1.303035221 | 1.841754 | 2.340907 | 1.24329498 | 2.8030352 |
| 53-C5-4-epi | A | 43.6909 1 | 30 2.5 98 | 5.479379 | 20.9884683 | 0.738731339 | 1.321980745 | 1.640391 | 2.480866 | 1.28317361 | 2.8219807 |
| 53-C5-1-epi | A | 56.3659 6 | 44 7.9 86 | 12.88553 | 22.9765859 | 1.110102444 | 1.361285498 | 1.751017 | 2.651264 | 1.33172526 | 2.8612855 |
| 53-C6-1-epi | A | 13.5582 8 | 25. 70 99 | 6.829803 | 3.52647013 | 0.834408167 | 0.54734021 | 1.132205 | 1.4101 | 0.97807965 | 2.0473402 |
| 53-C6-2-epi | A | 13.9567 8 | 54. 27 89 | 4.357219 | 4.66661956 | 0.639209422 | 0.669002397 | 1.144785 | 1.734631 | 1.07054848 | 2.1690024 |
| 53-C6-4-epi | A | 124. 769 1 | 78. 97 81 | 29.53675 | 34.2365956 | 1.470362682 | 1.534490573 | 2.096107 | 1.897507 | 1.11695668 | 3.0344906 |
| 53-C7-1-epi | A | 45.5649 3 | 17 6.8 94 | 6.460325 | 15.4695651 | 0.810254399 | 1.189478104 | 1.658631 | 2.247712 | 1.21674104 | 2.6894781 |
| 53-C7-3-epi | A | 41.4506 7 | 13 9.0 2 | 6.342314 | 12.1357918 | 0.802247771 | 1.084068118 | 1.617532 | 2.143077 | 1.18692715 | 2.5840681 |
| 53-C7-5-epi | A | 62.9975 2 | 47 5.1 79 | 10.26094 | 29.2495698 | 1.011186968 | 1.466119482 | 1.799323 | 2.676857 | 1.33901758 | 2.9661195 |
| 53-C8-1-epi | A | 40.114 | 51. 84 84 | 52.94606 | 10.9368242 | 1.72383364 | 1.038891231 | 1.603296 | 1.714736 | 1.06487962 | 2.5388912 |
| 53-C8-4-epi | A | 29.0215 4 | 71. 38 62 | 5.835087 | 8.56614703 | 0.766047354 | 0.932785524 | 1.46272 | 1.853614 | 1.10445047 | 2.4327855 |
| 53-C8-5-epi | A | 224. 047 5 | 11 3.6 84 | 5.207419 | 74.5358318 | 0.716622532 | 1.872365102 | 2.35034 | 2.055698 | 1.16203016 | 3.3723651 |
| 56-C1-5-epi | A | 54.0158 2 | 33 6.6 28 | 21.61002 | 23.3024836 | 1.334655109 | 1.367402211 | 1.732521 | 2.52715 | 1.29636131 | 2.8674022 |
| 56-C1-6-epi | A | 43.6433 6 | 36 1.8 34 | 18.49051 | 19.0077083 | 1.266948872 | 1.278929759 | 1.639918 | 2.55851 | 1.30529663 | 2.7789298 |
| 61-C1-1-epi | A | 13.2092 5 | 19. 65 74 | 51.32579 | 4.42989789 | 1.710335633 | 0.646393716 | 1.12878 | 1.293525 | 0.94486396 | 2.1463937 |
| 61-C5-3-epi | A | 77.9869 1 | 20 8.3 68 | 264. 4815 | 26.9659335 | 2.422395248 | 1.430815459 | 1.892022 | 2.318831 | 1.23700479 | 2.9308155 |
| 61-C5-4-epi | A | 51.4748 9 | 11 3.6 01 | 73.35411 | 19.2243164 | 1.865424455 | 1.283850906 | 1.711595 | 2.055384 | 1.16194072 | 2.7838509 |
| 61-C5-6-epi | A | 113. 291 2 | 31 7.6 61 | 197. 1129 | 38.5521643 | 2.294714981 | 1.586048764 | 2.054196 | 2.501963 | 1.28918491 | 3.0860488 |
| 61-C6-1-epi | A | 8.60962 5 | 7.703 84 | 19.72554 | 2.52515399 | 1.295029002 | 0.402287868 | 0.934984 | 0.886707 | 0.82894908 | 1.9022879 |
| 61-C6-2-epi | A | 14.6599 1 | 6.337 86 | 21.23214 | 4.11484067 | 1.326993824 | 0.614353024 | 1.166131 | 0.801943 | 0.80479702 | 2.114353 |
| 61-C6-3-epi | A | 264. 195 5 | 13 51. 54 | 290. 6632 | 111.403438 | 2.463389992 | 2.046898592 | 2.421925 | 3.130828 | 1.4683677 | 3.5468986 |
| 61-C6-4-epi | A | 6.59377 | 2.111 52 | 25.27627 | 3.01701318 | 1.402712961 | 0.479577207 | 0.819134 | 0.324596 | 0.66878635 | 1.9795772 |
| 61-C6-5-epi | A | 27.1657 2 | 1.956 3 | 8.913398 | 11.1658911 | 0.950043317 | 1.047893389 | 1.434021 | 0.291436 | 0.65933806 | 2.5478934 |
| 58-C5-1-epi | A | 143. 498 1 | 26. 30 91 | 167. 0647 | 37.4572561 | 2.222884572 | 1.573535961 | 2.156846 | 1.420106 | 0.98093054 | 3.073536 |
| 87-C1-6-ep | A | 244. 225 5 | 46 1.0 85 | 3.211763 | 89.1282936 | 0.506743509 | 1.950015592 | 2.387791 | 2.663781 | 1.33529182 | 3.4500156 |
| WS48-C01-01 epi | A | 134. 461 9 | 23 5.1 2 | 21.07618 | 41.5396 | 1.323791947 | 1.61846231 | 2.128599 | 2.37129 | 1.25195199 | 3.1184623 |
| WS48-C01-02 epi | A | 129. 602 3 | 17 8.4 38 | 22.75019 | 35.3961 | 1.356984991 | 1.548955413 | 2.112613 | 2.251487 | 1.21781641 | 3.0489554 |
| WS48-C01-03 epi | A | 83.5743 | 13 8.2 37 | 24.60165 | 28.4293 | 1.390964268 | 1.453766166 | 1.922073 | 2.140625 | 1.18622861 | 2.9537662 |
| WS48-C02-01 epi | A | 202. 592 1 | 37 7.4 29 | 24.4246 | 73.701 | 1.387827403 | 1.867473381 | 2.306623 | 2.576835 | 1.31051823 | 3.3674734 |
| WS48-C02-02 epi | A | 667. 246 7 | 31 7.8 88 | 9.720379 | 252.3594 | 0.9876832 | 2.402019486 | 2.824286 | 2.502274 | 1.28927333 | 3.9020195 |
| WS48-C02-03 epi | A | 90.8919 | 21 4.2 55 | 124. 7012 | 32.7472 | 2.095870605 | 1.515174172 | 1.958525 | 2.330931 | 1.24045259 | 3.0151742 |
| WS48-C03-01 epi | A | 290. 174 3 | 95 5.9 26 | 147. 0881 | 107.3257 | 2.167577664 | 2.03070373 | 2.462659 | 2.980424 | 1.42551302 | 3.5307037 |
| WS48-C03-02 epi | A | 174. 181 2 | 27 3.7 42 | 17.423 | 58.5972 | 1.241123051 | 1.767876864 | 2.210014 | 2.437341 | 1.27077195 | 3.2678769 |
| WS48-C03-03 epi | A | 10.3655 | 5.671 2 | 43.23874 | 3.5912 | 1.635873068 | 0.555239592 | 1.01559 | 0.753675 | 0.79104405 | 2.0552396 |
| WS48-C03-04 epi | A | 9.9792 | 3.994 4 | 58.68767 | 3.3801 | 1.768546856 | 0.528929549 | 0.999096 | 0.601452 | 0.74767095 | 2.0289295 |
| WS48-C04-01 epi | A | 42.1501 | 37. 36 02 | 30.405 | 12.3919 | 1.482945065 | 1.0931379 | 1.624799 | 1.572409 | 1.02432648 | 2.5931379 |
| WS48-C04-02 epi | A | 82.3263 | 93. 42 2 | 21.45209 | 25.3789 | 1.331469547 | 1.404472795 | 1.915539 | 1.970449 | 1.13774024 | 2.9044728 |
| WS48-C04-03 epi | A | 100. 792 | 26 9.2 53 | 38.5307 | 47.5185 | 1.58580694 | 1.676862723 | 2.003426 | 2.430161 | 1.26872626 | 3.1768627 |
| WS48-C04-04 epi | A | 180. 077 9 | 18 3.5 | 561. 1722 | 56.5159 | 2.749096169 | 1.752170648 | 2.25546 | 2.263636 | 1.22127809 | 3.2521706 |
| WS48-C04-05 epi | A | 24.1151 | 11. 04 87 | 14.51607 | 6.6079 | 1.161848962 | 0.820063462 | 1.382289 | 1.043311 | 0.87357027 | 2.3200635 |
| WS48-C06-04 epi | A | 358. 527 8 | 11 01. 67 | 53.36172 | 149.8471 | 1.727229831 | 2.175648342 | 2.554523 | 3.04205 | 1.44307222 | 3.6756483 |
| WS48-C07-01 epi | A | 28.4449 | 61. 36 87 | 92.26121 | 9.8639 | 1.965019141 | 0.994048661 | 1.454004 | 1.787947 | 1.08573977 | 2.4940487 |
| WS48-C07-03 epi | A | 91.4535 | 19 5.7 93 | 49.38913 | 30.3295 | 1.693631379 | 1.481865251 | 1.9612 | 2.291798 | 1.22930231 | 2.9818653 |
| WS48-C08-01 epi | A | 11.4503 | 6.583 8 | 38.9694 | 2.4333 | 1.590723696 | 0.386195656 | 1.058817 | 0.818477 | 0.80950803 | 1.8861957 |
| WS48-C08-03 epi | A | 24.7531 | 6.899 4 | 57.35282 | 8.3161 | 1.758554774 | 0.919919703 | 1.39363 | 0.838811 | 0.81530201 | 2.4199197 |
| WS48-C08-04 epi | A | 12.2831 | 10. 68 77 | 56.50124 | 3.1575 | 1.752057971 | 0.499343359 | 1.089308 | 1.028884 | 0.8694596 | 1.9993434 |
| WS48-C08-05 epi | A | 15.8959 | 16. 66 74 | 89.28206 | 4.0284 | 1.95076419 | 0.605132587 | 1.201285 | 1.221868 | 0.92444653 | 2.1051326 |
| WS60-C06-03 epi | A | 53.4836 | 30 2.1 54 | 28.76633 | 25.8598 | 1.458884408 | 1.412625162 | 1.728221 | 2.480228 | 1.28299176 | 2.9126252 |
| WS79-C01-01 epi | B | 0.879 | 7.067 | 403. 0813 | 0.2172 | 2.605392619 | -0.6631 40179 | -0.05 601 | 0.849235 | 0.81827206 | 0.8368598 |
| WS79-C01-02 epi | B | 1.4864 | 28. 88 87 | 242. 3181 | 0.6313 | 2.384385889 | -0.1997 64211 | 0.172136 | 1.460728 | 0.99250509 | 1.3002358 |
| WS79-C01-03 epi | B | 0.4371 | 8.078 6 | 175. 8814 | 0.1549 | 2.245219993 | -0.8099 48582 | -0.35 942 | 0.907336 | 0.83482681 | 0.6900514 |
| WS79-C01-04 epi | B | 2.3481 | 9.948 5 | 29.58268 | 0.5555 | 1.471037581 | -0.2553 15937 | 0.370717 | 0.997758 | 0.86059066 | 1.2446841 |
| WS79-C02-01 epi | B | 2.127 | 36. 96 15 | 81.08087 | 0.5243 | 1.908918412 | -0.2804 20142 | 0.32 7767 | 1.56775 | 1.02299882 | 1.2195799 |
| WS79-C02-02 epi | B | 0.1214 | 3.52 | 28.63321 | 0.1576 | 1.456870011 | -0.8024 43787 | -0.91 578 | 0.546543 | 0.73202572 | 0.6975562 |
| WS79-C02-05 epi | B | 4.896 | 17. 19 87 | 76.88723 | 1.3808 | 1.885854216 | 0.1401 30778 | 0.689841 | 1.235496 | 0.9283295 | 1.6401308 |
| WS79-C02-06 epi | B | 0.5909 | 0.378 7 | 67.18661 | 0.1122 | 1.827282742 | -0.9500 07143 | -0.22 849 | -0.42 17 | 0.45614243 | 0.5499929 |
| WS79-C03-01 epi | B | 0.059 | 9.122 4 | 483. 2182 | 0.0806 | 2.684143256 | | -1.22 915 | 0.960109 | 0.84986346 | 1.5 |
| WS79-C03-02 epi | B | 2.2147 | 5.649 9 | 115. 5935 | 0.5738 | 2.062933352 | -0.2412 39456 | 0.345315 | 0.752041 | 0.79057842 | 1.2587605 |
| WS79-C04-07 epi | B | 2.8181 | 12 0.8 88 | 92.10746 | 1.6659 | 1.964294793 | 0.221648928 | 0.44 9956 | 2.082385 | 1.16963408 | 1.7216489 |
| WS81-C01-01 epi | B | 3.0741 | 10. 30 66 | 581. 2596 | 0.6278 | 2.764370157 | -0.2021 78689 | 0.487718 | 1.013115 | 0.86496657 | 1.2978213 |
| WS81-C01-02 epi | B | 2.8634 | 33. 69 12 | 767. 7256 | 1.2557 | 2.885206022 | 0.098885894 | 0.45 6882 | 1.527516 | 1.01153517 | 1.5988859 |
| WS81-C01-03 epi | B | 4.3465 | 9.175 6 | 739. 5068 | 0.8279 | 2.868942169 | -0.0820 22117 | 0.63814 | 0.962634 | 0.85058301 | 1.4179779 |
| WS81-C02-07 epi | B | 1.96 | 5.014 | 1143 .691 | 0.4478 | 3.058308878 | -0.3489 15911 | 0.292256 | 0.700184 | 0.77580293 | 1.1510841 |
| WS81-C02-08 epi | B | 0.9523 | 3.509 8 | 606. 398 | 0.375 6 | 2.782757728 | -0.4252 74416 | -0.02 123 | 0.545282 | 0.73166663 | 1.0747256 |
| WS81-C04-06 epi | B | 9.6601 | 90. 19 79 | 218. 7291 | 3.9754 | 2.339906628 | 0.599380833 | 0.984982 | 1.955196 | 1.13339427 | 2.0993808 |
| WS81-C05-01 epi | B | 2.3974 | 2.993 5 | 649. 1371 | 0.6179 | 2.812336408 | -0.2090 81805 | 0.379741 | 0.476179 | 0.71197704 | 1.2909182 |
| WS81-C06-02 epi | B | 1.0797 | 4.573 6 | 101. 8272 | 0.2537 | 2.007863998 | -0.5956 79533 | 0.033303 | 0.660258 | 0.76442675 | 0.9043205 |
| WS81-C06-03 epi | B | 1.1129 | 1.772 7 | 526. 4002 | 0.3019 | 2.721316026 | -0.5201 36887 | 0.046456 | 0.248635 | 0.64714279 | 0.9798631 |
| WS81-C07-08 epi | B | 4.6781 | 3.224 6 | 2026 .804 | 1.2601 | 3.306811675 | 0.100405012 | 0.67007 | 0.508476 | 0.72117932 | 1.600405 |
| 80-C1-1-epi | B | 0.03976 3 | 1.579 74 | 59.38386 | 0.02917822 | 1.773668395 | -1.5349 41206 | -1.40 052 | 0.198586 | 0.63288215 | -0.03 4941 |
| 80-C1-2-epi | B | 0.23536 | 3.292 3 | 42.89979 | 0.10582801 | 1.632455163 | -0.9753 9937 | -0.62 827 | 0.517499 | 0.72375043 | 0.5246006 |
| 80-C1-3-epi | B | 0.01280 9 | 1.921 62 | 74.33559 | 0.00960878 | 1.871196786 | -2.0173 3175 | -1.89 249 | 0.28 3668 | 0.65712468 | -0.51 7332 |
| 80-C1-4-epi | B | 0.09026 | 8.484 29 | 86.67852 | 0.07751121 | 1.937911484 | -1.1106 35483 | -1.04 45 | 0.928615 | 0.84088995 | 0.3893645 |
| 80-C1-5-epi | B | 0.26 073 6 | 1.676 37 | 98.52178 | 0.02390402 | 1.993532242 | -1.6215 29056 | -0.58 38 | 0.22 4369 | 0.64022855 | -0.12 1529 |
| 80-C1-5-epi | B | 0.25 927 8 | 2.277 62 | 34.21617 | 0.06448994 | 1.534231332 | -1.1905 08027 | -0.58 623 | 0.35 7481 | 0.67815626 | 0.309492 |
| 80-C1-6-epi | B | 0.28 973 2 | 4.177 03 | 193. 115 | 0.08629815 | 2.285815973 | -1.0639 98514 | -0.53 8 | 0.620868 | 0.75320325 | 0.4360015 |
| 80-C2-2-ep | B | <0.0 033 756 5 | 0.132 2 | 90.53472 | 0.01445734 | 1.956815163 | -1.8399 11605 | | -0.87 876 | 0.325914 | -0.33 9912 |
| 80-C2-3-ep | B | 0.0418 | 0.071 99 | 78.6917 | 0.04911696 | 1.895928945 | -1.3087 68521 | -1.37 883 | -1.14 275 | 0.25069587 | 0.1912315 |
| 80-C3-1-epi | B | 0.09391 6 | 2.300 29 | 65.44897 | 0.06070618 | 1.8159028 | -1.2167 67095 | -1.02 726 | 0.361782 | 0.6793819 | 0.2832329 |
| 80-C3-3-epi | B | 0.15759 1 | 0.853 95 | 1031 .214 | 0.06283157 | 3.013348813 | -1.2018 22088 | -0.80 247 | -0.06 857 | 0.5567621 | 0.2981779 |
| 80-C3-4-epi | B | 0.11247 1 | 3.514 96 | 155. 4565 | 0.03554617 | 2.191608905 | -1.4492 07186 | -0.94 896 | 0.54592 | 0.73184832 | 0.0507928 |
| 80-C3-5-epi | B | <0.0 014 738 9 | 2.200 85 | 68.85405 | 0.02065146 | 1.837929497 | -1.6850 4924 | | 0.342591 | 0.6739137 | -0.18 5049 |
| 80-C3-6-epi | B | 0.16836 8 | 2.517 45 | 124. 5864 | 0.03110657 | 2.095470482 | -1.5071 47874 | -0.77 374 | 0.40 0962 | 0.69054522 | -0.00 7148 |
| 80-C3-7-epi | B | 0.18722 2 | 5.017 56 | 78.90903 | 0.07103849 | 1.897126703 | -1.1485 06279 | -0.72 764 | 0.700493 | 0.77589083 | 0.351 4937 |
| WS83-C06-01 epi | B | 0.3902 | 12. 62 17 | 86.33204 | 0.1582 | 1.936171981 | -0.8007 93521 | -0.40 871 | 1.101118 | 0.89004116 | 0.6992065 |
| WS83-C06-02 epi | B | 0.346 | 5.724 6 | 81.72381 | 0.0828 | 1.912348582 | -1.0819 69663 | -0.46 092 | 0.757745 | 0.79220377 | 0.4180303 |
| WS83-C06-03 epi | B | 0.0685 | 9.632 2 | 83.87686 | 0.0622 | 1.923642142 | -1.2062 09615 | -1.16 431 | 0.983725 | 0.85659248 | 0.2937904 |
| WS83-C06-04 epi | B | 0.0578 | 12. 42 25 | 86.41795 | 0.0117 | 1.936603969 | -1.9318 14138 | -1.23 807 | 1.094209 | 0.88807262 | -0.43 1814 |
| WS83-C06-05 epi | B | 1.4178 | 11. 20 18 | 145. 0466 | 0.5922 | 2.161507596 | -0.2275 31597 | 0.15 1615 | 1.049288 | 0.8752732 | 1.2724684 |
| WS83-C06-06 epi | B | 0.45 74 | 6.366 6 | 138. 719 | 0.1654 | 2.142136013 | -0.7814 64495 | -0.33 97 | 0.803908 | 0.80535686 | 0.7185355 |
| 82-C2-1ep | B | 0.04535 3 | 1.895 08 | 721. 852 | 0.02753711 | 2.858448155 | -1.5600 81641 | -1.34 339 | 0.277627 | 0.65540332 | -0.06 0082 |
| 82-C2-2ep | B | 0.38 951 2 | 4.651 91 | 190. 0249 | 0.1647162 | 2.278810455 | -0.7832 63685 | -0.40 948 | 0.667631 | 0.76652758 | 0.7167363 |
| 82-C3-1ep | B | 0.64389 3 | 3.380 92 | 245. 4284 | 0.55456912 | 2.38992483 | -0.2560 44317 | -0.19 119 | 0.529035 | 0.72703716 | 1.2439557 |
| 82-C3-2ep | B | 0.23287 2 | 2.897 44 | 114. 0148 | 0.01581336 | 2.056961338 | -1.8009 75842 | -0.63 288 | 0.46 2014 | 0.70794098 | -0.30 0976 |
| 82-C3-3ep | B | 0.01866 6 | 0.162 64 | 495. 66 | 0.01544216 | 2.695183842 | -1.8112 91952 | -1.72 894 | -0.78 877 | 0.35155432 | -0.31 1292 |
| 82-C3-4ep | B | 0.20150 5 | 5.094 8 | 164. 6249 | 0.05901913 | 2.216495561 | -1.2290 07197 | -0.69 571 | 0.707127 | 0.77778114 | 0.270 9928 |
| 82-C4-1ep | B | 0.09780 9 | 0.763 09 | 114. 8781 | 0.02048889 | 2.060237222 | -1.6884 81569 | -1.00 962 | -0.11 742 | 0.54284117 | -0.18 8482 |
| 82-C4-2ep | B | 0.02519 4 | 0.852 3 | 127. 224 | 0.03284234 | 2.104568947 | -1.4835 65907 | -1.59 87 | -0.06 941 | 0.55652204 | 0.0164341 |
| 82-C4-4ep | B | 0.58280 9 | 1.853 97 | 1008 .077 | 0.13467337 | 3.003493823 | -0.8707 18272 | -0.23 447 | 0.26 8104 | 0.65268992 | 0.6292817 |
| 82-C4-5ep | B | 5.47139 | 10. 73 88 | 609. 6532 | 1.26766808 | 2.785082835 | 0.103005555 | 0.738098 | 1.030956 | 0.87004978 | 1.6030056 |
| 82-C5-1ep | B | 0.08202 5 | 0.635 01 | 260. 5361 | 0.02846163 | 2.415867854 | -1.5457 40231 | -1.08 605 | -0.19 722 | 0.52010544 | -0.04 574 |
| 82-C5-2ep | B | 0.02214 | 0.069 1 | 230. 3408 | 0.00756326 | 2.362370915 | -2.1212 9097 | -1.65 483 | -1.16 05 | 0.24563724 | -0.62 1291 |

The ore prospecting method provided by the present invention has the advantages of short test time, low cost, convenience and efficiency, environmental protection, and capability of effectively shortening the mineral exploration period without damaging the environment, greatly improving the accuracy of rapid discrimination and target prediction of the ore prospecting type at the ore concentration area scale and reducing the exploration risk, which is a new and indispensable exploration means and method, and has an important promotion and popularization value.

In addition, application of a method for discriminating ore prospecting types based on compositional change of epidote above for the discrimination of an epithermal type Ag-Au deposit is provided, where the contents of La, Y, Gd, Yb, Sr and As elements in the obtained epidote are defined as V_{(La)}, V_{(Y)}, V_{(Gd)}, V_{(Yb)}, V_{(Sr)} and V_{(As)}, respectively.

(1) Calculation of a discriminant factor C1 is as follows:

performing logarithm transformation on the contents of La and Y elements in the obtained epidote to obtain lg(V_{(La)}) and lg(V_{(y)}), plotting with lg(V_{(La)}) as the abscissa and lg(V_{(Y)}) as the ordinate, respectively, obtaining a demarcation line between a porphyry type deposit and an epithermal type deposit based on the plotting range, and calculating the discriminant factor C1 by using the following formula:

$$\text{C1=0} \text{.28493059*lg} \left({\text{V}}_{\left(\text{La}\right)}\right) \text{+0} \text{.5762992}$$

(2) Calculation of a discriminant factor C2 is as follows:

performing logarithm transformation on the contents of Gd, Yb, Sr and As elements in the obtained epidote to obtain lg(V_{(Gd+Yb)}) and lg(V_{(Sr/As)}); plotting with the lg(V_{(Gd+Yb)}) as the abscissa and the lg(V_{(Sr/As)}) as the ordinate, obtaining a demarcation line between a porphyry type deposit and an epithermal type deposit based on the plotting range, and calculating the discriminant factor C2 by using the following formula:

$$\text{C2=lg} \left({\text{V}}_{\left(\text{Gd+Yb}\right)}\right) \text{+1} \text{.5}$$

(3) Discrimination of ore prospecting type is as follows:

substituting the content of La element in the obtained epidote into the formula above to calculate the discriminant factor C1, when C1 is less than lg(V_{(Y)}) and C2 is greater than lg(V_{(Sr/As)}), discriminating that a deposit to which the epidote belongs is the epithermal type deposit.

Application of a method for discriminating ore prospecting types based on compositional change of epidote above for the discrimination of a porphyry type Cu deposit is provided, where the contents of La, Y, Gd, Yb, Sr and As elements in the obtained epidote are defined as V_{(La)}, V_{(Y)}, V_{(Gd)}, V_{(Yb)}, V_{(Sr)} and Y_{(As)}, respectively.

(1) Calculation of a discriminant factor C1 is as follows:

performing logarithm transformation on the contents of La and Y elements in the obtained epidote to obtain lg(V_{(La)}) and lg(V_{(Y)}), plotting with lg(V_{(La)}) as the abscissa and lg(V_{(Y)}) as the ordinate, respectively, obtaining a demarcation line between a porphyry type deposit and an epithermal type deposit based on the plotting range, and calculating the discriminant factor C1 by using the following formula:

$$\text{C1=0} \text{.28493059*lg} \left({\text{V}}_{\left(\text{La}\right)}\right) \text{+0} \text{.5762992}$$

(2) Calculation of a discriminant factor C2 is as follows:

performing logarithm transformation on the contents of Gd, Yb, Sr and As elements in the obtained epidote to obtain lg(V_{(Gd+Yb)}) and lg(V_{(Sr/As)}); plotting with the lg(V_{(Gd+Yb)}) as the abscissa and the lg(V_{(Sr/As)}) as the ordinate, obtaining a demarcation line between a porphyry type deposit and an epithermal type deposit based on the plotting range, and calculating the discriminant factor C2 by using the following formula:

$$\text{C2=lg} \left({\text{V}}_{\left(\text{Gd+Yb}\right)}\right) \text{+1} \text{.5}$$

(3) Discrimination of an ore prospecting type is as follows:

substituting the content of La element in the obtained epidote into the formula above to calculate the discriminant factor C1, when C1 is greater than lg(V_{(Y)}) and C2 is less than lg(V_{(Sr/As)}), discriminating that a deposit to which the epidote belongs is the porphyry type deposit.

The foregoing embodiments merely express one or several embodiments of the present invention, the description is relatively specific and detailed, but cannot be construed as a limitation to the scope of the present invention. It should be pointed out that those of ordinary skilled in the art can make several transformations and improvements without departing from the concept of the present invention, which all belong to the scope of protection of the present invention.

What is claimed is:

1. A method for discriminating ore prospecting types based on compositional change of epidote, comprising the following steps:
metallogenic zone delineation: conducting data collection according to a selected research area, and comprehensively analyzing metallogenic potential to delineate a metallogenic favorable zone;
sample collection and analysis: collecting bedrock samples containing epidote from the metallogenic zone according to certain sampling units in step (1), wherein the density of sample points collected in the research area is 1-3 samples/Km2;
trace element analysis and testing: grinding the samples collected in step (2) into a probe sheet and a laser in-situ target, firstly observing corresponding epidote alteration characteristics of the probe sheet and the laser in-situ target under a microscope, and recording epidote alteration types in detail; then performing electron probe compositional analysis to determine the chemical composition and type of the epidote, and marking the chemical composition and type of the epidote; and carrying out in-situ microdomain element analysis of laser ablation inductively coupled plasma mass spectrometry (LA-ICPMS) on the mineral which is confirmed as the epidote by the electron probe composition, so as to obtain recorded data of each test point;
data processing and interpretation: processing the initial recorded data obtained in step (3) by using LADRlib software;
(5) ore prospecting type discrimination: processing data obtained in step (4) by using Excel, defining the contents of La, Y, Gd, Yb, Sr and As elements in the obtained epidote as V_{(La)}, V_{(Y)}, V_{(Gd)}, V_{(Yb)}, V_{(Sr)} and V_{(As)}, respectively, and substituting the content of La element in the obtained epidote into the following formula (1):
$$\text{C1=0} \text{.28493059*lg} \left({\text{V}}_{\left(\text{La}\right)}\right) \text{+0} \text{.5762992}$$
calculating a discriminant factor C1, when C1 is greater than lg(V_{(Y)}), discriminating that a deposit to which the epidote belongs is a porphyry type deposit; and when C1 is less than lg(V_{(Y)}), discriminating that a deposit to which the epidote belongs is an epithermal type deposit; and
substituting the contents of Gd and Yb elements in the obtained epidote into the following formula (2):
$$\text{C2=lg} \left({\text{V}}_{\left(\text{Gd+Yb}\right)}\right) \text{+1} \text{.5}$$
calculating a discriminant factor C2, when C2 is less than lg(V_{(Sr/As)}), discriminating that a deposit to which the epidote belongs is a porphyry type deposit; and when C2 is greater than lg(V_{(Sr/As)}), discriminating that a deposit to which the epidote belongs is an epithermal type deposit.

2. The method for discriminating ore prospecting types based on compositional change of epidote according to claim 1, wherein in step (5), the formula (1) is to perform logarithm transformation on the contents of La and Y elements in the epidote to obtain lg(V_{(La)}) and lg(V_{(Y)}), to plot with lg(V_{(La)}) as the abscissa and lg(V_{(Y)}) as the ordinate, respectively, and to obtain a demarcation line between the porphyry type deposit and epithermal type deposit based on the plotting range.

3. The method for discriminating ore prospecting types based on compositional change of epidote according to claim 1, wherein in step (5), the formula (2) is to perform logarithm transformation on the contents of Gd, Yb, Sr and As elements in the epidote to obtain lg(V_{(Gd+Yb)}) and lg(V_{(Sr/As)}), to plot with lg(V_{(Gd+Yb)}) as the abscissa and lg(V_{(Sr/As)}) as the ordinate, respectively, and to obtain a demarcation line between the porphyry type deposit and epithermal type deposit based on the plotting range.

4. The method for discriminating ore prospecting types based on compositional change of epidote according to claim 1, wherein step (2) comprises the following steps: positioning each sample point by using a GPS positioning system, collecting coordinate data X and Y, taking field photos, and making detailed field records to describe lithology, alteration and mineralization characteristics of each sample.

5. The method for discriminating ore prospecting types based on compositional change of epidote according to claim 1, wherein in step (4), the specific process is as follows:
(1) data import: importing the recorded data in a csv format obtained from an in-situ microdomain test point of each epidote sample into the LADRlib software in batches;
(2) data interpretation: obtaining a microdomain element integral curve of the sample at each observation point, and adjusting start time and end time of the integral curve of each observation point one by one according to the principle of ensuring the flattest and widest signal range of the selected element integral curve;
(3) data filtration: rejecting invalid data therein according to anomaly peaks of the element integral curve; and
(4) data export: summarizing interpreted and filtered data of each single-point microdomain, and then exporting the data in batch as a file in a csv format.

6. The method for discriminating ore prospecting types based on compositional change of epidote according to claim 1, wherein the research area is Zhunuo ore concentration area.

7. Application of a method for discriminating ore prospecting types based on compositional change of epidote according to claim 1 for the discrimination of an epithermal type Ag—Au deposit, wherein quantitative indexes of trace elements in the epidote are as follows: calculating discriminant factors C1=0.28493059*lg(V_{(La)})+0.5762992 and C2=lg(V_{(Gd+Yb)})+ 1.5, respectively, according to the formula (1) and the formula (2); and when C1 is less than lg(V_{(Y)}) and C2 is greater than lg(V_{(Sr+As)}), discriminating that a deposit to which the epidote belongs is an epithermal type deposit.

8. Application of a method for discriminating ore prospecting types based on compositional change of epidote according to claim 1 for the discrimination of a porphyry type Cu deposit, wherein quantitative indexes of trace elements in the epidote are as follows: calculating discriminant factors C1=0.28493059*lg(V_{(La)})+0.5762992 and C2=lg(V_{(Gd+Yb)})+ 1.5, respectively, according to the formula (1) and the formula (2), respectively; and when C1 is greater than lg(V_{(Y)}) and C2 is less than lg(V_{(Sr+as)}), discriminating that a deposit to which the epidote belongs is a porphyry type deposit.
